(19) United States
(12) Patent Application Publication
(10) Pub. No.: US 2004/0236242 A1
(43) Pub. Date: Nov. 25, 2004

(54) Capnograph system with integral controller

(76) Inventors: James E. Graham, Swampscott, MA (US); Robert K. O'Leary, Newton, MA (US); Dennis Witz, Georgetown, MA (US); David Vidal, Amesbury, MA (US)

Correspondence Address: Iandiorio & Teska, 260 Bear Hill Road, Waltham, MA, 02451-1018, US

(21) Appl. No.: 10/443,696

(22) Filed: May 22, 2003

(51) Int. Cl. A61B 5/08

(52) U.S. Cl. 600/532

(57) ABSTRACT

A capnograph system sensor head includes an airway adapter, a housing for receiving the airway adapter, a source of infrared radiation coupled to the housing for directing infrared radiation through the airway adapter, and a detector subsystem coupled to the housing and responsive to the infrared radiation after it passes through the airway adapter for providing an analog output. A circuit sub-assembly is integrated with the sensor head and includes a controller responsive to the analog output of the detector subsystem. The controller is configured to adjust the gain of the detector subsystem to output a digital signal representative of the amount of a particular gas flowing through the airway adapter.

### RELATED APPLICATIONS

This application claims priority to patent application Ser. No. 10/108,957 filed Mar. 28, 2002 and patent application Ser. No. 10/286,550 filed Nov. 1, 2002. All said applications are incorporated herein by this reference.

### FIELD OF THE INVENTION

This invention relates to a fluid concentration detection system, one particular species of which is a capnograph system.

### BACKGROUND OF THE INVENTION

Fluid (gas and liquid) concentration detection systems such as CO₂ gas analyzers, also called capnograph systems, are often used in the medical field and typically output a signal indicative of the concentration of CO₂ in a sample volume being monitored by the system.

In U.S. Pat. No. 5,616,923, incorporated herein by this reference, the CO₂ analyzer disclosed includes an emitter which directs a collimated beam of infrared radiation through a sample cell containing a gas sample and a detector including a “data” sensor and a reference sensor.

Infrared energy in a species specific band is absorbed by the gas of interest in the sample cell to an extent proportional to the concentration of that gas. Thereafter, the attenuated beam is directed to both the data sensor and the reference sensor. Band pass filters in front of those sensors limit the energy reaching them to specified different bands. Each of the sensors then outputs an electrical signal proportional in magnitude to the intensity of the energy striking that sensor.

Typically, the sensor head includes an infrared source for directing infrared radiation through an airway adapter connected to the patient and a detector which receives the infrared radiation and in response outputs an analog signal via a custom cable connected via a connector to a custom controller board fitted within a personal computer. The controller board and the computer software provided therewith process, digitize, and configure the analog signals output by the detector and then provide medical personnel with a readout showing the patient's CO₂ level.

Thus, the hospital typically purchases at least five separate components: the sensor head, the airway adapter, the controller board, the custom cable and connector, and the controller board software.

Technicians must install the controller board and the controlling software in the hospital's computer adding to the cost of the CO₂ gas analyzer. Moreover, the custom cable and connector are typically expensive costing forty dollars or more. And, the custom cable is susceptible to noise and also generates interfering emissions. In addition, currently available systems cannot be used in connection with laptop computers, handheld computers, or patient transport monitors due to the requirement of the separate controller board.

### SUMMARY OF THE INVENTION: I

It is therefore an object of this invention to provide a capnograph system in which the controller circuitry is uniquely integrated with the sensor head itself.

It is a further object of this invention to provide such a capnograph system which requires no separate controller board.

It is a further object of this invention to provide a capnograph sensor head which eliminates the need for a custom cable and connector.

It is a further object of this invention to provide such a capnograph system sensor head which is less susceptible to noise.

It is a further object of this invention to provide such a capnograph system sensor head which is less expensive.

It is a further object of this invention to provide such a capnograph system sensor head which is compact and lightweight.

It is a further object of this invention to provide such a capnograph system sensor head which does not generate interfering emissions.

It is a further object of this invention to provide such a capnograph system sensor head which can be used in connection with laptop computers, handheld computers, and patient transport monitors in addition to standard personal computers.

It is a further object of this invention to provide such a capnograph system sensor head which performs all the functions necessary to produce a digitized representation of a patient's CO₂ concentration directly within the sensor head.

It is a further object of this invention to provide such a capnograph system sensor head in which the head electronics are microprocessor based and require only external power to function.

It is a further object of this invention to provide such a capnograph system sensor head in which the data is presented in digital form via an RS232 compatible interface and in which the host interface incorporates a communication protocol to insure coherent information is passed between the host computer and the capnograph system sensor head.

The invention results from the realization that by integrating the controller of a capnograph system with the sensor head and programming it to automatically adjust the gain of the detector subsystem and then output a digital signal representative of the amount of CO₂ flowing through the airway adapter, the sensor head uniquely performs all the functions necessary to produce a digitized representation of the CO₂ concentration directly within the sensor head. The sensor head electronics are microprocessor based and require only external power to function and the CO₂ data is presented in digital form via a compatible interface. Also, the host interface incorporates a communication protocol to ensure coherent information is passed between both devices. Placing the electronics package right at the sensor head provides an improved signal to noise ratio, improved source control, and the flexibility to implement a variety of signal conditioning schemes when deemed beneficial. The microprocessor allows for flexibility in programming as well as dynamic adjustment of operation based on variable conditions during operation. Thus, the device is not fixed in one mode of operation as is the situation with the prior art. The microprocessor functionality also typically includes the ability to store and retrieve device specific operating parameters. This makes the device capable of handling manufacturing tolerances as well as issues that arise from component aging and varying operating conditions throughout the device lifespan. The microprocessor programming typically also includes functions for automatically controlling the source power, for adjusting the sensor gains, signal conditioning algorithms that can be selectively applied, for monitoring the device input voltage, as well as detection and consequential action when errors are detected.

From a safety perspective, this approach is superior to a remote hardware implementation since the control resides right at the sensing circuitry. This feature enables the device to detect, respond, and alert the host computer to error conditions. The response is immediate and can place the device in a safe mode when necessary to protect the device against damage and the patient from erroneous data. The host may also make determinations regarding error conditions and instruct the device to respond accordingly. Furthermore, the proximity of the controlling electronics to the sensor head including the source and the detector subsystem provides the most reliable interface.

This invention results from the further realization that the need for and the problems associated with a beam splitter in CO₂ gas analyzers and other fluid concentration detection systems can be eliminated by the use of an integrating lens in the detector positioned to integrate the collimated radiation passing through the airway adapter evenly over the sample sensor and reference sensor of the detector subsystem so that the instantaneous fields of view of the sample sensor and the reference sensor are the same to equalize any obscuration effects thereof to thus provide a more compact, less expensive, lower power, and highly sensitive capnograph system.

This invention results from the still further realization that a much simpler, inexpensive, and reversible airway adapter apparatus is effected, in the preferred embodiment, by a gas analyzer housing with a mortise extending between first and second end walls both having a lengthy outwardly facing depression on each side of the mortise and an airway adapter with a tenon which fits in the mortise of the housing and which has an outwardly extending ears each with a lengthy inwardly facing detent which snap fits into a depression on the housing irrespective of the orientation of the airway adapter to releasably retain the airway adapter in the housing without ball and spring mechanisms or clips or the like.

This invention features a capnograph system sensor head with an airway adapter, a housing for receiving the airway adapter, a source of infrared radiation coupled to the housing for directing infrared radiation through the airway adapter, and a detector subsystem coupled to the housing and responsive to the infrared radiation after it passes through the airway adapter for providing an analog output. A circuit sub-assembly is uniquely integrated with the sensor head, typically the housing, and the circuit sub-assembly includes a controller responsive to the analog output of the detector subsystem. The controller is configured to adjust the gain of the detector subsystem and configured to output a digital signal representative of the amount of a particular gas flowing through the airway adapter.

The integrated circuit sub-assembly is preferably disposed on a flex circuit folded and received by the housing. The controller may be programmed to adjust the optical output level of the source in response to the output level of the detector subsystem. Typically, the circuit subassembly further includes an amplifier connected between the controller and the source. In one example, the amplifier is a field effect transistor.

The controller may be programmed to amplify the output of the detector subsystem in response to the output level of the detector subsystem. Thus, the detector subsystem typically includes an amplification circuit responsive to the controller. Preferably, the controller is programmed, in response to the output level of the detector subsystem, to both adjust the optical output level of the source and to amplify the output level of the detector subsystem.

The invention further includes a cable connected on one end to the housing for transmitting the digital signal and the circuit subassembly typically further includes a communications chip connected between the controller and the cable. In one example, the communications chip is configured to convert a TTL signal output by the controller to an RS 232 compatible digital signal. The cable then includes a distal connector. Also, the circuit subassembly may include a memory having calibration coefficients for the source and the detector subsystem stored therein. In one example, the memory is an EE PROM. The circuit subassembly may further include a voltage regulation circuit configured to provide a reference voltage and to protect the circuit subassembly against over voltage conditions and a logic circuit connected between the detector subsystem and the controller. The logic circuit typically includes a channel responsive to a reference sensor of the detector subsystem and a channel responsive to the sample sensor of the detection subsystem. One preferred controller includes a processor responsive to both channels and an analog-to-digital converter.

The preferred detector subsystem includes a sample sensor, a reference sensor, and an integrating lens positioned to integrate collimated radiation passing through the airway adapter evenly over the sample sensor and the reference sensor so that the instantaneous field of view of the sample sensor and the reference sensor are the same to minimize any obscuration effects thereof.

The preferred source includes a radiation source and a collimating lens which forms a collimated beam. Typically, the collimating lens is positioned at a distance from the radiation source such that the radiation source is completely imaged by the collimating lens. The collimating lens has a focal length greater than the distance between the collimating lens and the radiation source. In one example, the radiation source is an infrared radiation producing filament, the collimating lens is one half of a sapphire ball lens, the flat surface of which faces the radiation source.

Typically, the integrating lens of the detector is positioned at a distance from the sample sensor and the reference sensor such that the sample sensor and the reference sensor are both completely imaged by the integrating lens. Preferably, the integrating lens has a focal length greater than the distance between the integrated lens and the sample and reference sensors. One integrating lens is one half of a sapphire ball lens, the flat surface of which faces the sample and reference detectors.

The preferred source also includes a TO header, a filament supported above the header, a TO can mated with the TO header and including an aperture therein, and a collimating lens positioned in the can between the filament and the aperture. The preferred detector subsystem may then include a header having a reference sensor and a sample sensor mounted thereon adjacent each other, a filter pack above the reference and sample sensors, and a TO can mounted with the header and including an aperture therein, and an integrating lens positioned in the TO can between the aperture therein and the filter pack.

In one example, the source includes a header, a filament supported above the header, a can mated with the header and including an aperture therein, and a collimating lens positioned in the can between the filament and the aperture which outputs a collimated beam of radiation across the airway adapter. One possible detector subsystem includes a header having a reference sensor and a sample sensor mounted thereon adjacent each other, a filter pack above the reference sensor and sample sensors, a TO can mounted with the header and including an aperture therein, and an integrating lens positioned in the TO can between the aperture therein and the filter pack to integrate the collimated radiation passing through the airway adapter evenly over the sample sensor and the reference sensor so that the instantaneous fields of view of the sample sensor and the reference sensor are the same to equalize any obscurations effects thereof.

One preferred housing includes first and second spaced end walls, a mortise extending from the first end wall to the second wall, and one of a detent and a depression on at least one of said end walls. One preferred airway adapter includes tubular end portions, a tenon there between received in the mortise of the housing, and at least one ear including the other of the detent and the depression for releasably locking the airway adapter in the housing.

Typically, both the first and second spaced end walls of the housing include a depression on each side of the mortise, all the depressions are longer than they are wide, and there are two opposing ears, one on each side of the tenon, each ear including a detent longer then it is wide. The tenon then includes spaced opposing side walls and there is an ear extending outwardly from a proximal end of each side wall, a ledge extending outwardly from the top of each side wall, is an end wall extending outwardly from the distal end of each side wall. Each end wall also includes the other of the detent and the depression. There are also end walls each extending outwardly from the proximal end of each side wall, each said end wall spaced behind an ear. Each side wall has an orifice therein and each orifice preferably includes a circumferential seat. A window in each seat covers the orifice and the window is treated with an anti-fogging compound. The mortise then includes spaced side walls each including an orifice aligned with the orifices in the side walls of the tenon and the junction between the side walls of the mortise of the housing and the end walls of the housing are chamfered. In one example, the airway adapter is made of a rigid plastic material such as polystyrene. Typically, the housing is made of metal such as aluminum. In one specific example, the invention features a capnograph system sensor head with an airway adapter and a housing for receiving the airway adapter. The preferred housing includes first and second end walls, a mortise extending from the first end wall to the second wall and one of a detent and a depression on at least one of said end walls. A preferred source of radiation coupled to the housing for directing radiation through the airway adapter includes a header, a filament supported above the header, a TO can mated with the header and including an aperture therein, and a collimating lens positioned in the can between the filament and the aperture which outputs a collimated beam of radiation across the airway adapter. A preferred detector subsystem coupled to the housing and responsive to the radiation after it passes through the airway adapter for providing an analog output includes a header having a reference sensor and a sample sensor mounted thereon adjacent each other, a filter pack above the reference sensor and sample sensors, a TO can mounted with the header and including an aperture therein, and an integrating lens positioned in the TO can between the aperture therein and the filter pack to integrate the collimated radiation passing through the airway adapter evenly over the sample sensor and the reference sensor so that the instantaneous fields of view of the sample sensor and the reference sensor are the same to equalize any obscurations effects thereof. A circuit sub-assembly is integrated with the sensor head and includes a controller responsive to the analog output of the detector subsystem, the controller configured to adjust the gain of the detector subsystem and configured to output a digital signal representative of the amount of a particular gas flowing through the airway adapter.

A preferred capnograph system sensor head in accordance with this invention features a housing for receiving an airway adapter, a source of radiation coupled to the housing for directing radiation through the airway adapter, a detector subsystem coupled to the housing and responsive to the radiation after it passes through the airway adapter for providing an analog output, an integrated circuit sub-assembly disposed on a flex circuit folded and received by the housing, the circuit sub-assembly including a controller responsive to the analog output of the detector subsystem, the controller configured to adjust the gain of the detector subsystem and configured to output a digital signal representative of the amount of a particular gas flowing through the airway adapter, and a cable connected on one end to the integrated circuit sub-assembly for transmitting the digital signal, the circuit sub-assembly further including a communications chip connected between the controller and the cable.

### BRIEF DESCRIPTION OF THE DRAWINGS

Other objects, features and advantages will occur to those skilled in the art from the following description of a preferred embodiment and the accompanying drawings, in which:

FIG. 1 is a block diagram showing the primary components associated with a typical prior art capnograph system;

FIG. 2 is a partial schematic view showing the unique airway adapter and housing portions of the capnograph system sensor head of the subject invention;

FIG. 3 is another schematic view showing the infrared radiation source, the detector subsystem, and a circuit assembly configured on a flex circuit as a component of the capnograph system sensor head of the subject invention in addition to the housing and airway adapter portion shown in FIG. 2;

FIG. 4 is a top view of the flex circuit shown in FIG. 3;

FIG. 5 is a more detailed circuit diagram showing the primary components associated with the circuit subassembly of this invention including the microcontroller disposed on the flex circuit shown in FIGS. 3 and 4 integrated with the sensor head;

FIG. 6 is a schematic cross sectional exploded view showing one preferred source of infrared radiation for the capnograph system sensor head of the subject invention; and

FIG. 7 is a schematic three dimensional exploded view showing the primary components associated with one preferred detector subsystem of the subject invention.

### DISCLOSURE OF THE PREFERRED EMBODIMENT

Aside from the preferred embodiment or embodiments disclosed below, this invention is capable of other embodiments and of being practiced or being carried out in various ways. Thus, it is to be understood that the invention is not limited in its application to the details of construction and the arrangements of components set forth in the following description or illustrated in the drawings.

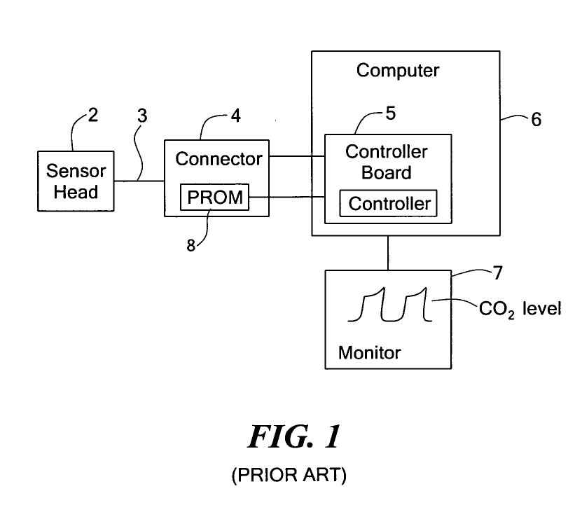

As discussed in the background section above, prior art capnograph system 1, FIG. 1 includes sensor head 2 with an airway adapter and a source of infrared radiation and a detector (not shown) coupled to custom cable 3 connected via custom connector 4 to controller board 5 fitted inside personal computer 6 which is connected to monitor 7. One supplier provides PROM 8 in connector 4 for storing calibration constants unique to each sensor head 2. As discussed in the background section above, controller board 5 and the computer software associated with it process and then digitize the analog signals output by the detector of sensor head 2 and provide medical personnel with a readout as shown on monitor 7 representing the patient's CO₂ level.

Unfortunately, hospital personnel must typically purchase at least five separate components from the manufacturer: sensor head 2, the airway adapter associated with it, controller board 5, custom cable 3 and connector 4, and the controller board software. Technicians must then install controller board 5 and the controller board software in the hospital's computer 6 adding to the cost of the CO₂ gas analyzer system. Moreover, custom cable 3 and connector 4 are typically expensive costing $40.00 or more. In addition, custom cable 3 is susceptible to noise and also generates interfering emissions. Also, currently available systems as shown in FIG. 1 cannot be used in connection with laptop computers, handheld computers, or patient transport monitors due to the requirement of separate controller board 5.

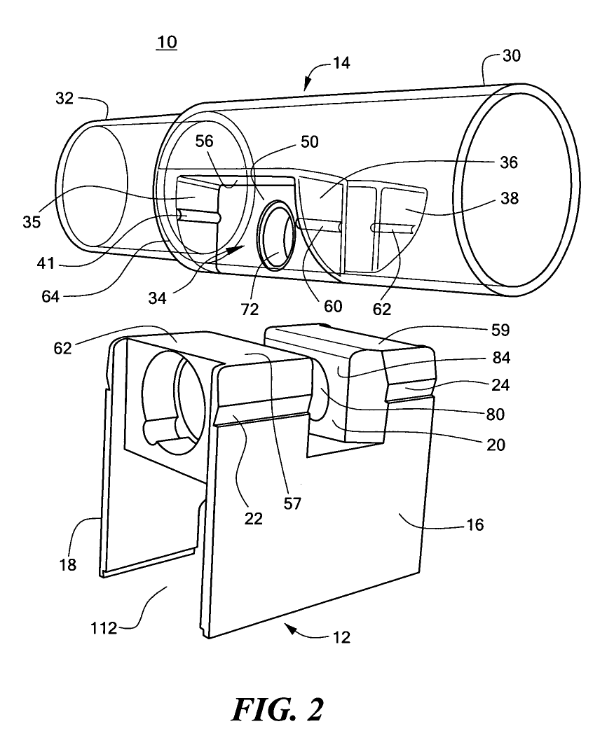

In the subject invention, in contrast, capnograph system sensor head 10, FIG. 2 includes airway adapter 14 and housing 12 for receiving airway adapter 14. In the preferred embodiment, housing 12 includes first 16 and second 18 end walls and mortise 20 extending from first end wall 16 to second end wall 18. One but preferably both end walls 16 and 18 include lengthy, narrow, outwardly facing depressions 22 and 24 on each side of mortise 20 as shown for end wall 16.

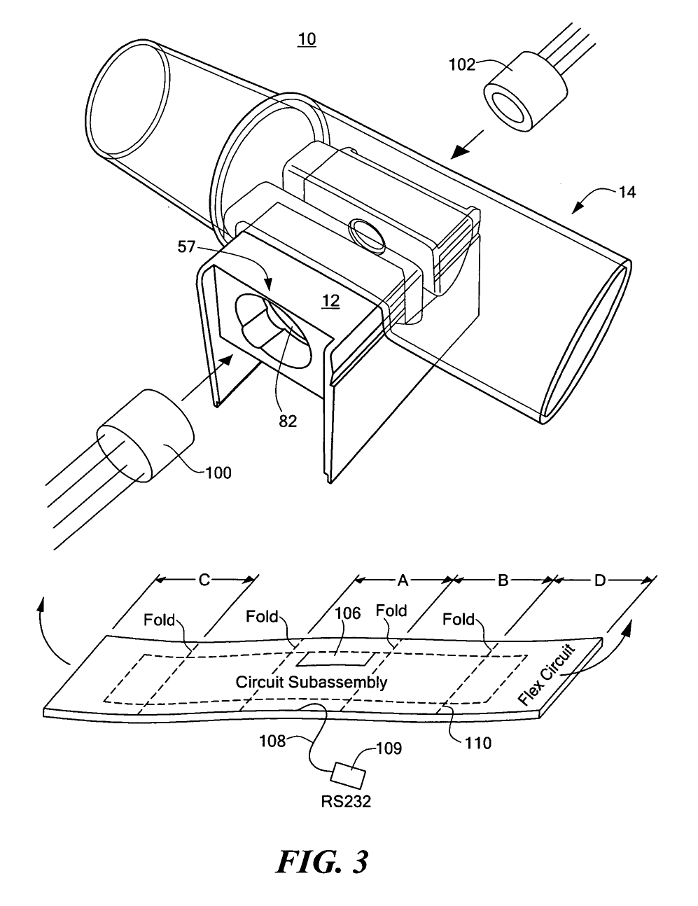

Airway adapter 14 includes tubular end portions 30, 32 and tenon 34 therebetween received in mortise 20 of housing 12 as shown in FIG. 3. In the preferred embodiment, airway adapter 14 also includes ears 35 and 36 both including lengthy, narrow, inwardly facing detents such as detent 41 on ear 35 for releasably locking and retaining airway adapter 14 in housing 12 in a precise manner and orientation. In other embodiments, however, the detents may be on the walls of the housing and the depressions located in the ears of the airway adapter but it is preferred that the depressions be located in the walls of the housing to prevent wear.

The tenon of the airway adapter preferably includes spaced opposing side walls such as side wall 50 and ears such as ear 35 which extend outwardly from the proximal end of each tenon side wall. Ledges, such as ledge 56, extend outwardly from the top of each tenon side wall. The ledges rest on top surfaces 57, 59 of housing 12. Airway adapter 14 also preferably includes end walls such as end wall 36 each extending outwardly from the distal end of each tenon side wall as shown for tenon side wall 50. The end walls also preferably each include inwardly facing detents such as detent 60 and the detents are received in depressions 22 and 24 of housing end wall 16. Additional end walls such as end wall 64 each extend outwardly from the proximal end of each tenon side wall spaced behind their respective ears as shown.

Each tenon side wall 50 includes an orifice: orifice 72 as shown for side wall 50. A circumferential seat receives a plastic window preferably treated with an anti-fog compound as discloses in U.S. Pat. No. 6,095,986. In other examples, the whole of the airway adapter may be treated with an anti-fog treatment after the windows are secured to their respective orifice seats. The seats ensure the windows do not actually touch any portion of the housing to prevent scratching of the windows.

Orifice 72 and the window covering it, and the opposite orifice and window, are aligned with the orifices (see orifice 80) in the spaced side walls of mortise 20 of housing 12. Airway adapter 14 is preferably symmetrical about axis A.

A source of infrared radiation 100, FIG. 3 is coupled to the housing 12 and transmits infrared radiation through one orifice, through the window covering the corresponding orifice of airway adapter 14, through the window covering the opposite orifice thereof, and to detector subsystem 102 coupled to the opposing orifice in the housing 12. This arrangement can be reversed, however. Air from the patient flows through the space between the tenon side walls and adapter 14 which is enclosed by the sidewalls and the top cylindrical wall and bottom cylindrical wall of airway adapter 14.

Airway adapter 14 is preferably made of a rigid plastic material such as polystyrene. Housing 12 is typically made of metal such as aluminum. The windows are preferably made of polystyrene.

In one specific example, airway adapter 14 is 2.375 inches long. The tubular end portion 30 outside diameter tapers from 0.679 to 0.718 inches, while the inside diameter tapers from 0.609 to 0.571 inches. The tubular end portion 32 outside diameter tapers from 0.599 to 0.618 inches, while the inside diameter tapers from 0.529 to 0.508 inches. Housing 12 is typically about 1.1 inches long and 0.6 inches wide and mortise 20 is typically about 0.191 inches wide and 0.348 inches deep.

The benefits of this preferred arrangement is that airway adapter 14, FIGS. 2-3 is inexpensive to manufacture, easy to use, reversible, light weight, compact, and can be manufactured at a low cost. Other airway adapters/housing combinations or configurations, however, are possible in accordance with this invention.

Detector subsystem 102, FIG. 3, is responsive to infrared radiation output by source 100 after it passes through airway adapter 14 and provides an analog output.

In the subject invention, as discussed above, circuit sub-assembly 104 is uniquely integrated with sensor head 10. Circuit subassembly 104 includes means such as controller 106 (e.g., a microprocessor) responsive to the analog output of detector 102 and configured to adjust the gain of the detector subsystem 102 and thereafter output a digital signal representative of the concentration of CO₂ flowing through airway adapter 14 via digital cable 108 fitted with a typical RS 232 connector or a specially configured connector.

In the preferred embodiment, integrated circuit subassembly 104 including controller 106 is disposed on flex circuit 110. Flex circuit 110 is folded and received in channel 112, FIG. 2 of housing 12. The typical fold lines are shown in FIG. 3. Area A is folded on top of area B. Area C and D include the electrical contacts for source 100 and detector subsystem 102, respectively, and fold up so that sensor 100 can be disposed in orifice 82 of housing 12 while detector subsystem 102 is disposed in the opposite orifice thereof.

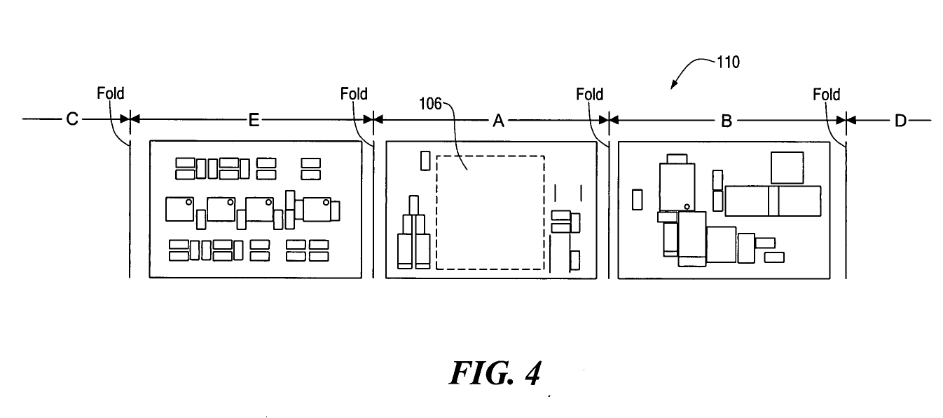

FIG. 4 shows flex circuit 110 in more detail. Controller 106, typically a microprocessor as discussed above with analog-to-digital conversion and optionally programmable gate array capabilities and functionality is disposed as shown on the underside of flex circuit board section A. Section A is then folded onto section B placing section E on top of section A with ears C and D folded up.

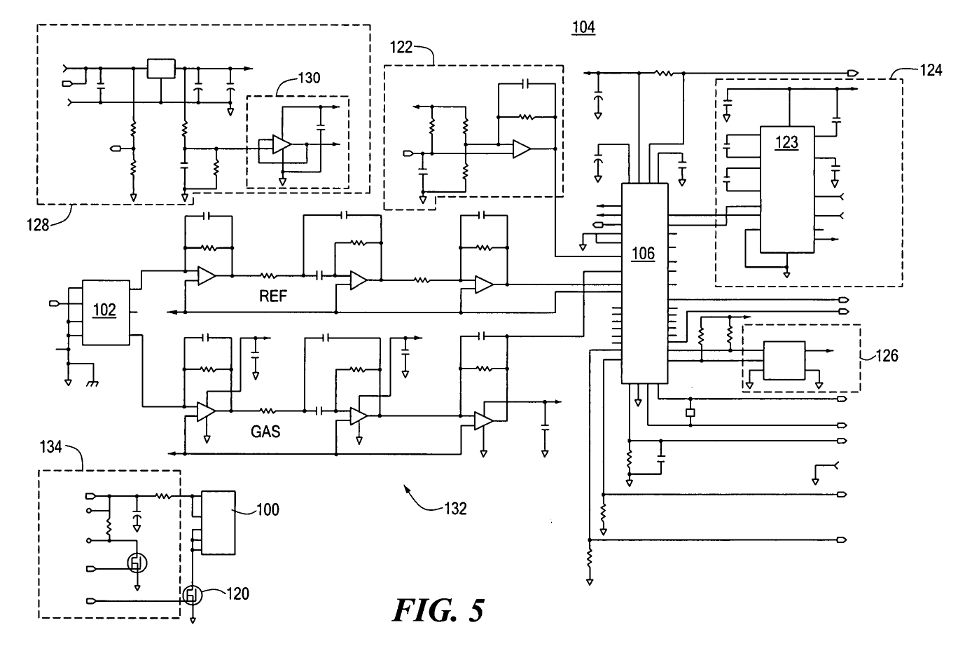

Subassembly 104, FIG. 5 is preferably configured so that controller 106 can adjust the optical output level of source 100 in response to the output level of detector subsystem 102. That is, source 100 is driven by controller 106 based on the output level of detector subsystem 102 by an amplifier, preferably field effect transistor 120 connected between source 100 and controller 106. It is also possible in addition or alternatively to amplify the output of the detector subsystem in response to its previous output via detector amplification circuit 122 responsive to controller 106. Typically, amplification circuit 122 is housed within detector subsystem 102, FIG. 3.

In this way, controller 106 in conjunction with field effect transistor 120 and/or amplification circuit 122 adjusts the gain of detector subsystem 102.

Communications chip 123 and its related circuitry 124 is connected between controller 106 and digital cable 108, FIG. 3 and converts the digital TTL signal output by controller 106 representative of the CO₂ level detected by detector subsystem 102 to an RS 232 compatible digital signal.

EE PROM memory 126 stores the calibration coefficients for the particular detector/source combination. Voltage regulation circuit 128 includes reference voltage generator 130 configured to provide a reference voltage and to protect the circuit subassembly against over voltage conditions. Logic circuit 132 connected between detector subsystem 102 and controller 106 typically includes two channels as shown: reference and gas channels configured such that the reference channel is responsive to the reference sensor of the detector subsystem and a gas channel responsive to the gas or data sensor of the detection subsystem. Optional heating circuitry 134 for source 100 is also shown in FIG. 5.

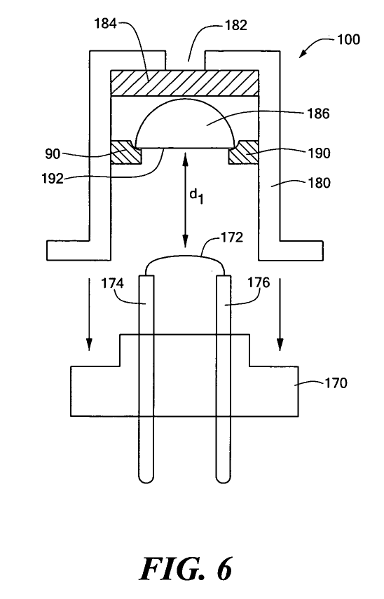

The preferred infrared radiation source device 100, FIG. 6 includes TO type header 170 and 0.070 inch long by 0.070 inch wide serpentine infrared radiation producing tungsten filament 172 supported above header 170 by electrodes 174 and 176 connected to the power source circuitry 128 shown in FIG. 5. The impedance of filament 172 is optimally designed to match the impedance of this power source (for example, 9 Ohms) connected to electrodes 174 and 176. TO can 180 is mated and hermetically sealed with respect to header 170 and includes aperture 182 in the top thereof as shown. Optional sapphire window element 184 seals aperture 182 with respect to TO can 180.

Collimating lens 186 is positioned between filament 172 and aperture 182 at a distance d₁ from filament 172 such that filament 172 is completely imaged by collimating lens 186. Collimating lens 186 is held in place inside TO can 180 via holder 190. In one example, distance d₁ was 60 mils. In the same example, collimating lens 186 was one half of a sapphire ball lens and had a focal length slightly greater then distance d₁. As shown, flat surface 192 of the half ball lens faces filament 172 to collimate the infrared radiation produced thereby for transmission out through aperture 182 and through airway adapter 14, FIGS. 2-3. Other applicable radiation source devices include the emitter shown in the '923 patent as well as filament and are gas type radiation producers incorporating an optical element or elements which, at least to some extent, collimate the radiation. Examples of other applicable optical elements include the use of reflector or plano convex lenses.

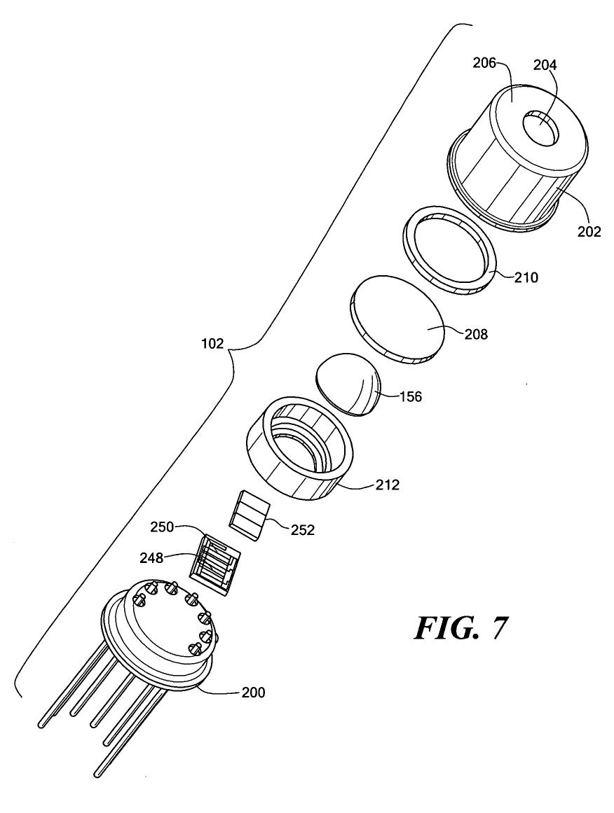

In the preferred embodiment, the other half of the sapphire ball lens is used as integrating lens 156, FIG. 7 of detector subsystem 102. Detector subsystem 102, in this example, includes TO header 200 having reference sensor 250 and sample sensor 248 mounted adjacent each other thereon. Filter pack 252 is located right above the sensors. TO can 202 is hermetically sealed with respect to header 200 and includes aperture 204 in top surface 206 thereof which receives the attenuated collimated beam after it passes through the airway adapter. Inside TO can 202 is sapphire window 208 behind seal 210 which seals aperture 204 with respect to can 202. Behind window 208 is integrating lens 156 held in place by lens holder 212 between aperture 204 and filter pack 252.

The adjacent active areas of PbSe sensors 248 and 250 conveniently lie in the same plane and integrating lens 156 is positioned at a distance thereof such that both the sample 248 and reference 250 sensors are completely imaged by integrating lens 156. Preferably, the focal length of integrating lens 156 is slightly greater than the distance between integrating lens 156 and the sample and reference detectors so that the instantaneous field of view of the sample sensor and the reference sensor are the same to equalize any obscuration effects thereof. As shown, the flat surface of the half ball lens faces the sample and reference detectors. The output from reference sensor 250 is coupled to the reference channel of logic circuit 132, FIG. 5 and the output of sample sensor 248, FIG. 7 is coupled to the gas channel of logic circuit 132, FIG. 5 before being digitized and processed by controller 106. In other embodiments, the filter materials (coatings) and the sensors may be configured as set forth in the '923 patent or as known in the art.

In this way, the controlling electronics for the capnograph system are integrated with the sensor head and the controller thereof is programmed to adjust the gain of the detector subsystem and output a digital signal representative of the amount of CO₂ flowing through the airway adapter. Thus, the sensor head is able to perform all of the functions necessary to produce a digitized signal in contrast to an analog representation of the CO₂ concentration directly within the sensor head. The sensor head electronics are microprocessor based and require only external power to function. The CO₂ data is conveniently presented in digital form via an RS 232 compatible interface. The computer hosting the interface may incorporate a communication protocol and in this way coherent information is shared as it is passed between both devices. By placing the electronics package right at the sensor head, the signal to noise ratio is improved as is control of the infrared radiation source. Moreover, there is now a unique ability to flexibly implement a variety of signal conditioning schemes when deemed beneficial by the manufacturer. The integrated controller allows for flexibility and programming as well as dynamic adjustment of operation based on variable conditions during operation of the capnograph. Unlike the prior art, the device is not fixed in one mode of operation. Also, the controller functionality can now include the ability to store and retrieve device specific operating parameters which makes the device capable of handling of manufacturing in tolerances as well as issues that arise from component aging and operating conditions throughout the device life span. The controller is uniquely programmed to automatically control the power supply to the infrared radiation source to adjust the gain or gains of the detector subsystem to invoke signal conditioning algorithms that can be selectively applied, to monitor device input voltage, and to take corrective action when errors are detected.

From a safety perspective, this approach is far superior to the remote hardware implementation shown in FIG. 1 since controller 106, FIGS. 3-5 resides right with the sensing circuitry of circuit subassembly 104 which itself is integrated with housing 12. In this way, the device is able to detect, respond, and alert the host computer to error conditions. The response is immediate and can place the device in a safe mode when necessary to protect the device against damage and also to protect the patient from erroneous data. The host can make determinations regarding error conditions and instruct the device to respond accordingly. Furthermore, the proximity of the controlling electronics to the sensor and detector provides the most reliable interface. The resulting sensor head is small and compact and also lightweight and there is no need for a separate controller board which must be installed by technicians thus reducing the price of the capnograph system. Custom cables and connectors are not required further reducing the cost of the system. Finally, the unique sensor head of the subject invention with the integrated controller can now be used in connection with laptop computers, handheld computers, and even patient transport monitors because a separate controller board is not required.

Although specific features of the invention are shown in some drawings and not in others, this is for convenience only as each feature may be combined with any or all of the other features in accordance with the invention. The words “including”, “comprising”, “having”, and “with” as used herein are to be interpreted broadly and comprehensively and are not limited to any physical interconnection. Moreover, any embodiments disclosed in the subject application are not to be taken as the only possible embodiments.

Other embodiments will occur to those skilled in the art and are within the following claims:

What is claimed is:

1. A capnograph system sensor head comprising:
an airway adapter;
a housing for receiving the airway adapter;
a source of radiation coupled to the housing for directing radiation through the airway adapter;
a detector subsystem coupled to the housing and responsive to the radiation after it passes through the airway adapter for providing an analog output; and
a circuit sub-assembly integrated with the sensor head, the circuit sub-assembly including a controller responsive to the analog output of the detector subsystem, the controller configured to adjust the gain of the detector subsystem and configured to output a digital signal representative of the amount of a particular gas flowing through the airway adapter.

2. The sensor head of claim 1 in which the integrated circuit sub-assembly is disposed on a flex circuit folded and received by the housing.

3. The sensor head of claim 1 in which the controller is programmed to adjust the optical output level of the source in response to the output level of the detector subsystem.

4. The sensor head of claim 3 in which the circuit subassembly further includes an amplifier connected between the controller and the source.

5. The sensor head of claim 4 in which the amplifier is a field effect transistor.

6. The sensor head of claim 1 in which the controller is programmed to amplify the output of the detector subsystem in response to the output level of the detector subsystem.

7. The sensor head of claim 6 in which the detector subsystem includes an amplification circuit responsive to the controller.

8. The sensor head of claim 1 in which the controller is programmed, in response to the output level of the detector subsystem, to both adjust the optical output level of the source and to amplify the output level of the detector subsystem.

9. The sensor head of claim 1 further including a cable connected on one end to the housing for transmitting the digital signal.

10. The sensor head of claim 9 in which the circuit subassembly further includes a communications chip connected between the controller and the cable.

11. The sensor head of claim 10 in which the communications chip is configured to convert a TTL signal output by the controller to a compatible digital signal.

12. The sensor head of claim 9 in which the cable includes a distal connector.

13. The sensor head of claim 1 in which the circuit subassembly further includes a memory having calibration coefficients for the source and the detector subsystem stored therein.

14. The sensor head of claim 13 in which the memory is an EE PROM.

15. The sensor head of claim 1 in which the circuit subassembly further includes a voltage regulation circuit configured to provide a reference voltage and to protect the circuit subassembly against over voltage conditions.

16. The sensor head of claim 1 in which the circuit subassembly further includes a logic circuit connected between the detector subsystem and the controller.

17. The sensor head of claim 16 in which the logic circuit includes a channel responsive to a reference sensor of the detector subsystem and a channel responsive to the sample sensor of the detection subsystem.

18. The sensor head of claim 17 in which the controller includes a processor responsive to both channels.

19. The sensor head of claim 17 in which the controller includes an analog-to-digital converter.

20. The sensor head of claim 1 in which the detector subsystem includes:
a sample sensor,
a reference sensor, and
an integrating lens positioned to integrate collimated radiation passing through the airway adapter evenly over the sample sensor and the reference sensor so that the instantaneous field of view of the sample sensor and the reference sensor are the same to equalize any obscuration effects thereof.

21. The sensor head of claim 1 in which the source includes:
a radiation source; and
a collimating lens which forms a collimated beam.

22. The sensor head of claim 21 in which the collimating lens is positioned at a distance from the radiation source such that the radiation source is completely imaged by the collimating lens.

23. The sensor head of claim 22 in which the collimating lens has a focal length greater than the distance between the collimating lens and the radiation source.

24. The sensor head of claim 21 in which the radiation source is an infrared radiation producing filament.

25. The sensor head of claim 21 in which the collimating lens is one half of a ball lens, the flat surface of which faces the radiation source.

26. The sensor head of claim 25 in which the collimating lens is made of sapphire.

27. The sensor head of claim 20 in which the integrating lens is positioned at a distance from the sample sensor and the reference sensor such that the sample sensor and the reference sensor are both completely imaged by the integrating lens.

28. The sensor head of claim 27 in which the integrating lens has a focal length greater than the distance between the integrated lens and the sample and reference sensors.

29. The sensor head of claim 20 in which the integrating lens is one half of a ball lens, the flat surface of which faces the sample and reference detectors.

30. The sensor head of claim 29 in which the integrating lens is made of sapphire.

31. The sensor head of claim 1 in which the source includes a TO header, a filament supported above the header, a TO can mated with the TO header and including an aperture therein, and a collimating lens positioned in the can between the filament and the aperture.

32. The sensor head of claim 1 in which the detector subsystem includes a TO header having a reference sensor and a sample sensor mounted thereon adjacent each other, a filter pack above the reference and sample sensors, and a TO can mounted with the header and including an aperture therein, and an integrating lens positioned in the TO can between the aperture therein and the filter pack.

33. The sensor head of claim 1 in which the source includes a header, a filament supported above the header, a can mated with the header and including an aperture therein, and a collimating lens positioned in the can between the filament and the aperture which outputs a collimated beam of radiation across the airway adapter and wherein the detector subsystem includes a header having a reference sensor and a sample sensor mounted thereon adjacent each other, a filter pack above the reference sensor and sample sensors, a TO can mounted with the header and including an aperture therein, and an integrating lens positioned in the TO can between the aperture therein and the filter pack to integrate the collimated radiation passing through the airway adapter evenly over the sample sensor and the reference sensor so that the instantaneous fields of view of the sample sensor and the reference sensor are the same to equalize any obscurations effects thereof.

34. The sensor head of claim 1 in which the housing includes:
first and second spaced end walls,
a mortise extending from the first end wall to the second wall, and
one of a detent and a depression on at least one of said end walls.

35. The sensor head of claim 34 in which the airway adapter includes:
tubular end portions,
a tenon therebetween received in the mortise of the housing, and
at least one ear including the other of the detent and the depression for releasably locking the airway adapter in the housing.

36. The sensor head of claim 35 in which both the first and second spaced end walls of the housing include a depression on each side of the mortise.

37. The sensor head of claim 36 in which all the depressions are longer than they are wide.

38. The sensor head of claim 37 in which there are two opposing ears, one on each side of the tenon, each ear including a detent longer then it is wide.

39. The sensor head of claim 35 in which the tenon includes spaced opposing side walls.

40. The sensor head of claim 39 in which there is an ear extending outwardly from a proximal end of each side wall.

41. The sensor head of claim 39 further including a ledge extending outwardly from the top of each side wall.

42. The sensor head of claim 39 in which there is an end wall extending outwardly from the distal end of each side wall.

43. The sensor head of claim 42 in which each said end wall also includes the other of the detent and the depression.

44. The sensor head of claim 40 in which there are end walls each extending outwardly from the proximal end of each side wall, each said end wall spaced behind an ear.

45. The sensor head of claim 39 in which each side wall has an orifice therein.

46. The sensor head of claim 45 in which each orifice includes a circumferential seat.

47. The sensor head of claim 46 further including a window in each seat covering the orifice.

48. The sensor head of claim 47 in which the window is treated with an anti-fogging compound.

49. The sensor head of claim 45 in which the mortise includes spaced side walls each including an orifice aligned with the orifices in the side walls of the tenon.

50. The sensor head of claim 49 in which the junction between the side walls of the mortise of the housing and the end walls of the housing are chamfered.

51. The sensor head of claim 1 in which the airway adapter is made of a rigid plastic material.

52. The sensor head of claim 51 in which said rigid plastic material is polystyrene.

53. The sensor head of claim 1 in which the housing is made of metal.

54. The sensor head of claim 53 in which said metal is aluminum.

55. A capnograph system sensor head comprising:
a housing for receiving an airway adapter;
a source of infrared radiation coupled to the housing for directing infrared radiation through the airway adapter;
a detector subsystem coupled to the housing and responsive to the infrared radiation after it passes through the airway adapter for providing an analog output; and
a circuit sub-assembly integrated with the sensor head, the circuit sub-assembly including a controller responsive to the analog output of the detector subsystem, the controller configured to adjust the gain of the detector subsystem and configured to output a digital signal representative of the amount of a particular gas flowing through the airway adapter.

56. A capnograph system sensor head comprising:
an airway adapter;
a housing for receiving the airway adapter;
a source of radiation coupled to the housing for directing radiation through the airway adapter;
a detector subsystem coupled to the housing and responsive to the radiation after it passes through the airway adapter for providing an analog output; and
a circuit sub-assembly integrated with the sensor head, the circuit sub-assembly including means responsive to the analog output of the detector subsystem, for adjusting the gain of the detector subsystem and outputting a digital signal representative of the amount of a particular gas flowing through the airway adapter.

57. The sensor head of claim 56 in which the integrated circuit sub-assembly is disposed on a flex circuit folded and received by the housing.

58. The sensor head of claim 56 further including a cable connected on one end to the housing for transmitting the digital signal.

59. The sensor head of claim 59 in which the circuit subassembly further includes a communications chip connected between the controller and the cable.

60. The sensor head of claim 56 in which the circuit subassembly further includes a memory having calibration coefficients for the source and the detector subsystem stored therein.

61. The sensor head of claim 60 in which the memory is a EE PROM.

62. A capnograph system sensor head comprising:
an airway adapter;
a housing for receiving the airway adapter;
a source of radiation coupled to the housing for directing radiation through the airway adapter;
a detector subsystem coupled to the housing and responsive to the radiation after it passes through the airway adapter for providing an analog output, the detector subsystem including:
a sample sensor,
a reference sensor, and
an integrating lens positioned to integrate the collimated radiation passing through the airway adapter evenly over the sample sensor and the reference sensor so that the instantaneous field of view of the sample sensor and the reference sensor are the same to minimize any obscuration effects thereof; and
a circuit sub-assembly integrated with the housing, the circuit sub-assembly including a controller for processing the analog output.

63. A capnograph system sensor head comprising:
an airway adapter;
a housing for receiving the airway adapter;
a source of radiation coupled to the housing for directing radiation through the airway adapter, the source including:
a radiation source, and
a collimating lens which forms a collimated beam of radiation, the collimating lens is positioned at a distance from the radiation source such that the radiation source is completely imaged by the collimating lens;
a detector subsystem coupled to the housing and responsive to the collimated beam after it passes through the airway adapter for providing an analog output; and
a circuit sub-assembly integrated with the sensor head, the circuit sub-assembly including a controller for processing the analog output.

64. A capnograph system sensor head comprising:
an airway adapter;
a housing for receiving the airway adapter;
a source of radiation coupled to the housing for directing radiation through the airway adapter, the source including:
a header, a filament supported above the header, a TO can mated with the header and including an aperture therein, and a collimating lens positioned in the can between the filament and the aperture which outputs a collimated beam of radiation across the airway adapter;
a detector subsystem coupled to the housing and responsive to the collimated beam after it passes through the airway adapter for providing an analog output, the detector subsystem including a header having a reference sensor and a sample sensor mounted thereon adjacent each other, a filter pack above the reference sensor and sample sensors, a TO can mounted with the header and including an aperture therein, and an integrating lens positioned in the TO can between the aperture therein and the filter pack to integrate the collimated radiation passing through the airway adapter evenly over the sample sensor and the reference sensor so that the instantaneous fields of view of the sample sensor and the reference sensor are the same to minimize any obscuration effects thereof; and
a circuit sub-assembly integrated with the sensor head, the circuit sub-assembly including a controller responsive to the analog output of the detector subsystem, the controller configured to adjust the gain of the detector subsystem and configured to output a digital signal representative of the amount of a particular gas flowing through the airway adapter.

65. A capnograph system sensor head comprising:
an airway adapter;
a housing for receiving the airway adapter, the housing including:
first and second spaced end walls, a mortise extending from the first end wall to the second wall, and one of a detent and a depression on at least one of said end walls;
a source of radiation coupled to the housing for directing radiation through the airway adapter;
a detector subsystem coupled to the housing and responsive to the radiation after it passes through the airway adapter for providing an analog output; and
a circuit sub-assembly integrated with the housing, the circuit sub-assembly including a controller for processing the analog output.

66. A capnograph system sensor head comprising:
an airway adapter;
a housing for receiving the airway adapter, the housing including:
first and second end walls, a mortise extending from the first end wall to the second wall and one of a detent and a depression on at least one of said end walls,
a source of radiation coupled to the housing for directing radiation through the airway adapter, the source including:
a header, a filament supported above the header, a TO can mated with the header and including an aperture therein, and a collimating lens positioned in the can between the filament and the aperture which outputs a collimated beam of radiation across the airway adapter;
a detector subsystem coupled to the housing and responsive to the radiation after it passes through the airway adapter for providing an analog output, the detector subsystem including:
a header having a reference sensor and a sample sensor mounted thereon adjacent each other, a filter pack above the reference sensor and sample sensors, a TO can mounted with the header and including an aperture therein, and an integrating lens positioned in the TO can between the aperture therein and the filter pack to integrate the collimated radiation passing through the airway adapter evenly over the sample sensor and the reference sensor so that the instantaneous fields of view of the sample sensor and the reference sensor are the same to minimize any obscuration effects thereof; and
a circuit sub-assembly integrated with the sensor head, the circuit sub-assembly including a controller responsive to the analog output of the detector subsystem, the controller configured to adjust the gain of the detector subsystem and configured to output a digital signal representative of the amount of a particular gas flowing through the airway adapter.

67. A capnograph system sensor head comprising:
a housing for receiving an airway adapter;
a source of radiation coupled to the housing for directing radiation through the airway adapter;
a detector subsystem coupled to the housing and responsive to the radiation after it passes through the airway adapter for providing an analog output;
an integrated circuit sub-assembly disposed on a flex circuit folded and received by the housing, the circuit sub-assembly including a controller responsive to the analog output of the detector subsystem, the controller configured to adjust the gain of the detector subsystem and configured to output a digital signal representative of the amount of a particular gas flowing through the airway adapter; and
a cable connected on one end to the integrated circuit sub-assembly for transmitting the digital signal, the circuit subassembly further including a communications chip connected between the controller and the cable.
